(19) United States
(12) Patent Application Publication
(10) Pub. No.: US 2020/0281486 A1
(43) Pub. Date: Sep. 10, 2020

(54) BLOOD PRESSURE CUFF HAVING IMPROVED COMFORT AND SAFETY AND METHODS OF MANUFACTURING SAME

(71) Applicant: Angelica Raquel Johnson, Orinda, CA (US)

(72) Inventor: Angelica Raquel Johnson, Orinda, CA (US)

(21) Appl. No.: 16/798,901

(22) Filed: Feb. 24, 2020

Related U.S. Application Data
(60) Provisional application No. 62/815,757, filed on Mar. 8, 2019.

(51) Int. Cl. A61B 5/022; A61B 5/00

(52) U.S. Cl. CPC A61B 5/02233; A61B 2560/04; A61B 5/6824

(57) ABSTRACT

A blood pressure cuff extending between a proximal end and a distal end includes a body having a fillable chamber and a hose connector, the body having a pair of opposing sides having at least one first pair of concave curvatures formed approximately ⅓ along a length of the body from the proximal end to the distal end.

### RELATED APPLICATIONS

The present application claims priority to U.S. Provisional Patent Application Ser. No. 62/815,757, filed Mar. 8, 2019, the contents of which is hereby incorporated by reference in its entirety as if fully set forth herein.

### FIELD OF THE DISCLOSURE

The present disclosure relates to blood pressure cuffs for use in medical settings. More particularly the present disclosure relates to blood pressure cuffs having novel profiles for improved fit, comfort, and safety.

### BACKGROUND OF THE DISCLOSURE

Cuffs for measuring blood pressure generally include a tubular constrictable sleeve for a limb of a person, a source for fluid pressure, means for measuring static pressure, and means arranged in proximity to an artery for listening to flow in the artery.

Modern blood pressure measurements have long traditions and fall into two distinct types. Both the auscultatory and the oscillometric method use the constriction of an artery to such a degree that blood flow is stopped and then allowed to flow while a signal derived from the blood pressure is monitored. The constriction occurs by means of a cuff surrounding a limb (in most cases an upper arm or a wrist). The cuff has a non-stretchable fabric on the outside enclosing an elongate bladder surrounding a large part of the limb periphery. The bladder is pressurized by means of air, and the air pressure is monitored. The Korotkoff method depends on listening to sounds in the artery downstream of the constriction as blood begins to flow, and to read the pressure when certain sounds related to the heartbeat are heard and again when sounds begin to disappear. Traditionally, the listening has occurred by means of a stethoscope, the chestpiece of which is held against the skin in proximity to the artery downstream from the occlusion, frequently supported against the edge of the cuff.

Traditional cuffs are generally rectangular in shape and tend to fit poorly. These cuffs may be prone to error. For example, if the cuff is too small the blood pressure readings may be artefactually high. Alternatively, if the cuff is too big the blood pressure readings will be too low. The cuff often slips down past the patient's antecubital fossa causing skin tears, blisters, ecchymosis and unnecessary pain. Additionally, rectangular cuffs require a nurse or practitioner to reapply the cuff frequently for patient comfort and safety. In an ICU setting, for example, vital signs are taken frequently depending on the condition of the patient. Patients may require blood pressure readings at five, ten, fifteen or thirty-minute intervals. While most cuffs are disposable, they are often kept for longer durations, and they may become discolored, soiled or emit a foul odor, harboring bacteria.

The present disclosure seeks to address many of these problems in order to improve patient safety and comfort.

### SUMMARY OF THE DISCLOSURE

In some embodiments, a blood pressure cuff extending between a proximal end and a distal end includes a body having a fillable chamber and a hose connector, the body having a pair of opposing sides having at least one first pair of concave curvatures formed approximately ⅓ along a length of the body from the proximal end to the distal end.

### BRIEF DESCRIPTION OF THE DISCLOSURE

Various embodiments of the presently disclosed blood pressure cuffs are disclosed herein with reference to the drawings, wherein:

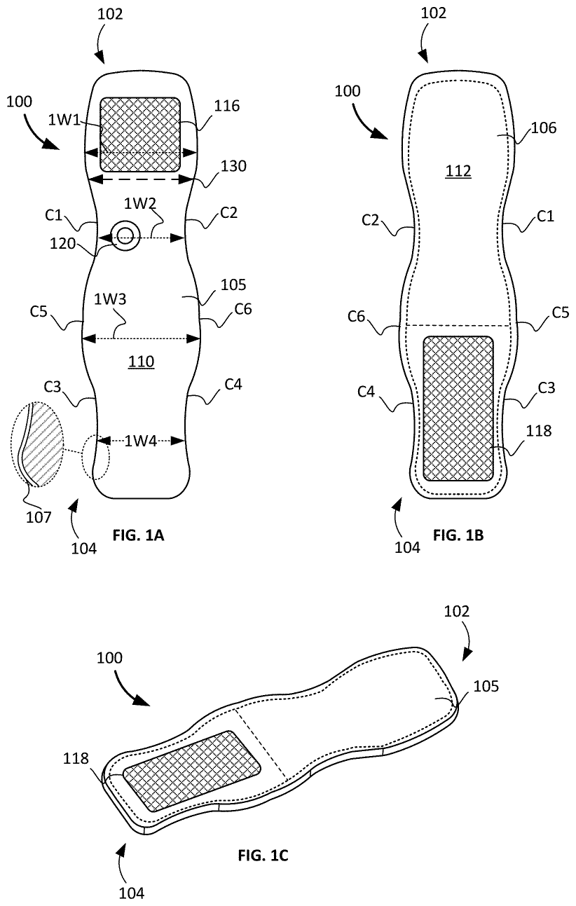

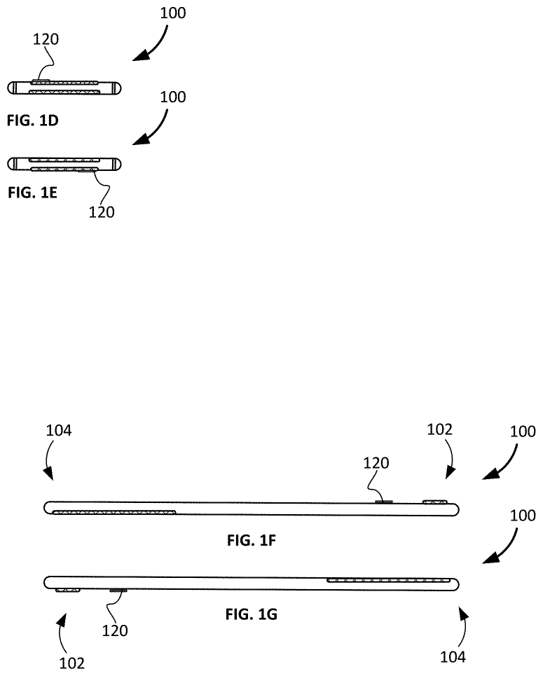

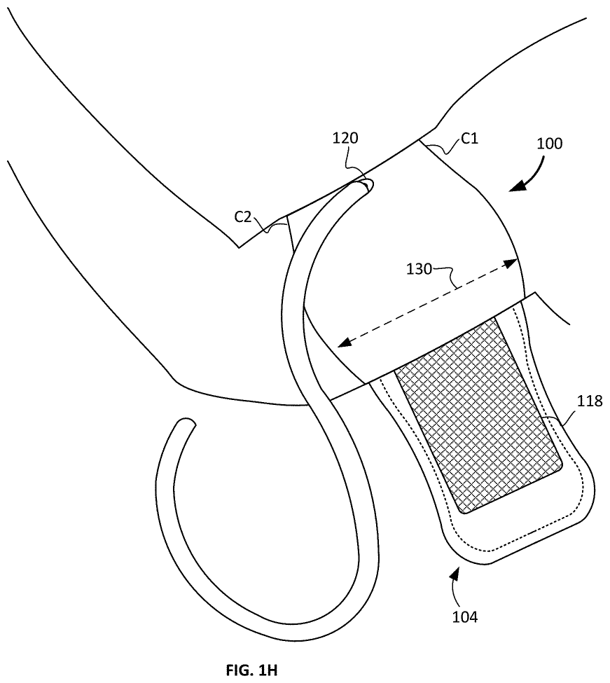

FIGS. 1A-G are schematic bottom, top, perspective, front, back, and side views of a blood pressure cuff according to a first embodiment of the present disclosure;

FIG. 1H is a schematic illustration of the blood pressure cuff of FIGS. 1A-G being disposed about a patient's arm;

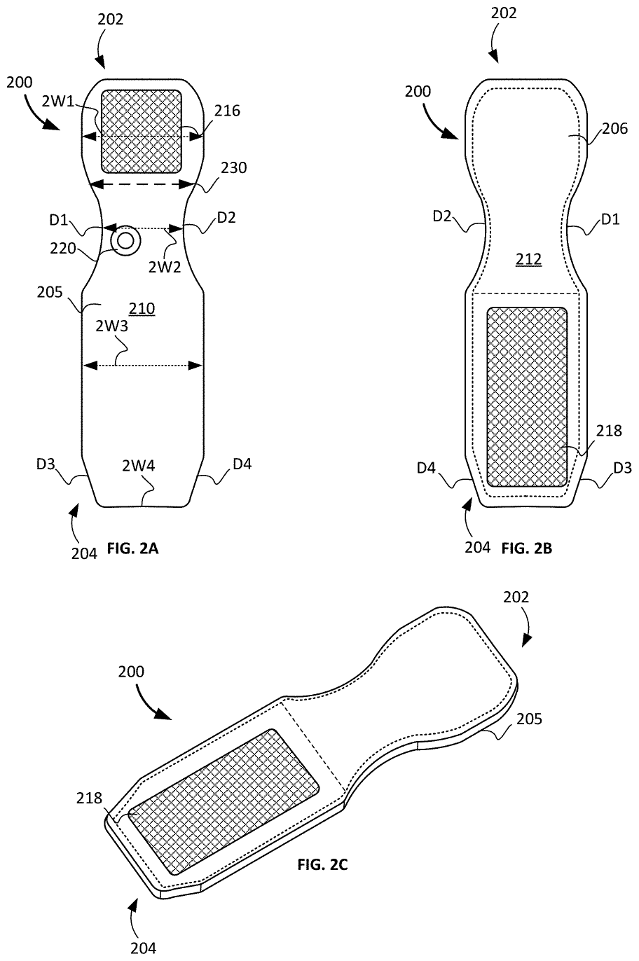

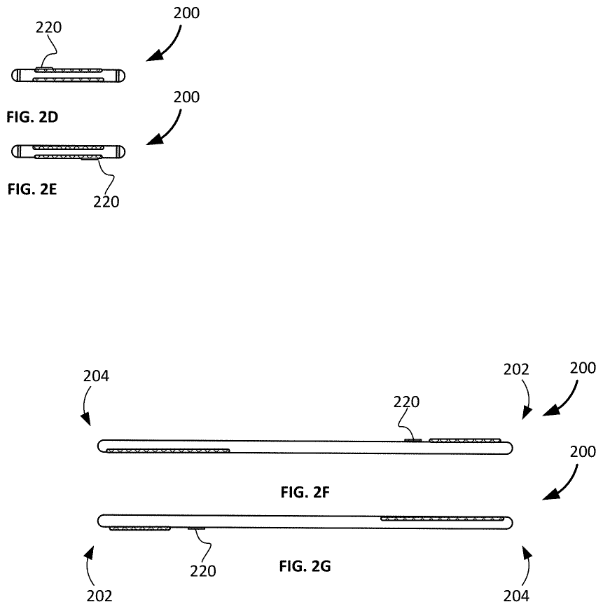

FIGS. 2A-G are schematic bottom, top, perspective, front, back, and side views of a blood pressure cuff according to a second embodiment of the present disclosure;

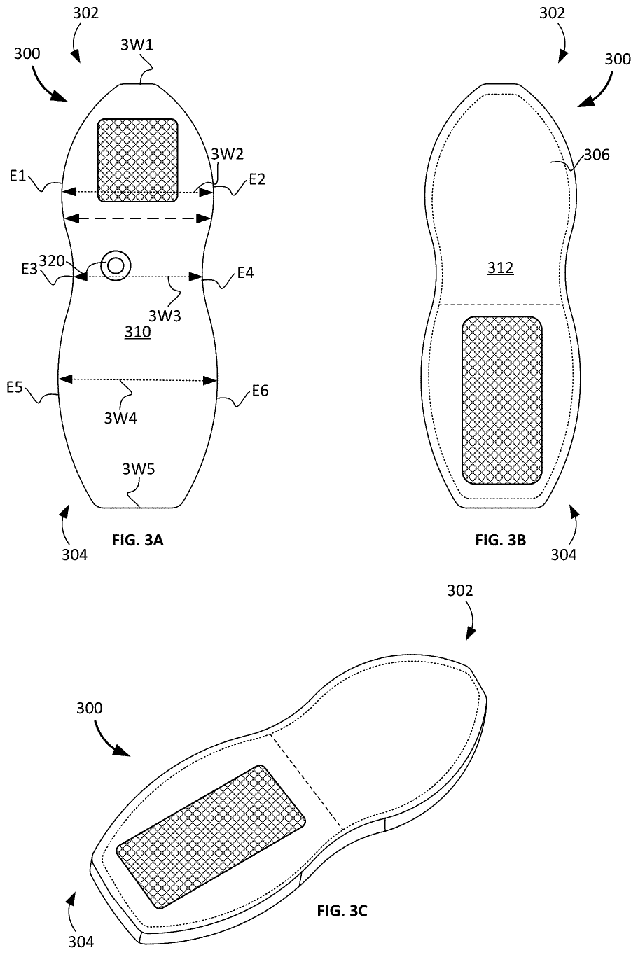

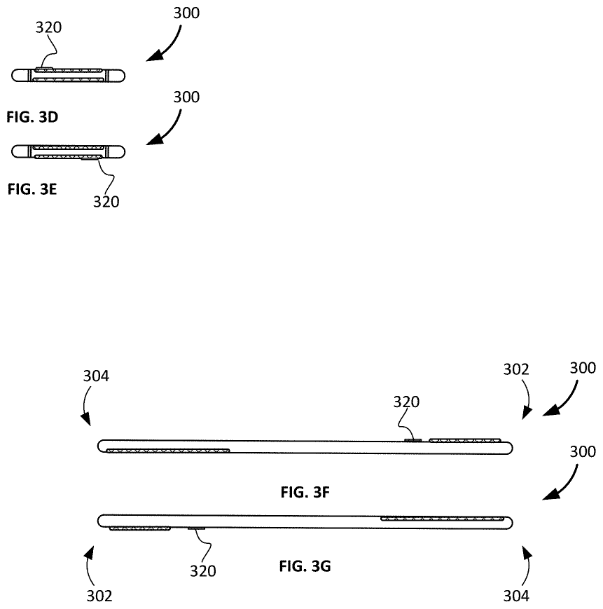

FIGS. 3A-G are schematic bottom, top, perspective, front, back, and side views of a blood pressure cuff according to a third embodiment of the present disclosure;

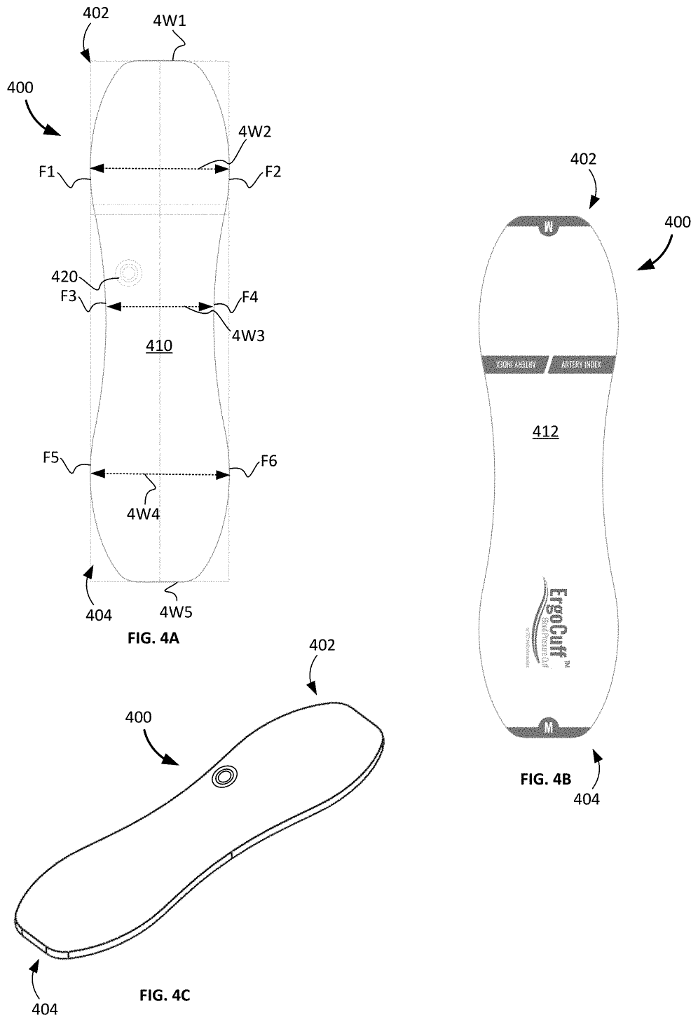

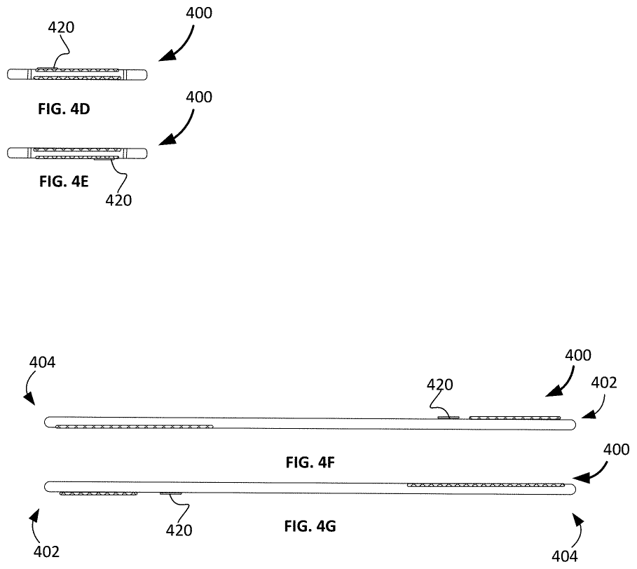

FIGS. 4A-G are schematic bottom, top, perspective, front, back, and side views of a blood pressure cuff according to a fourth embodiment of the present disclosure; and

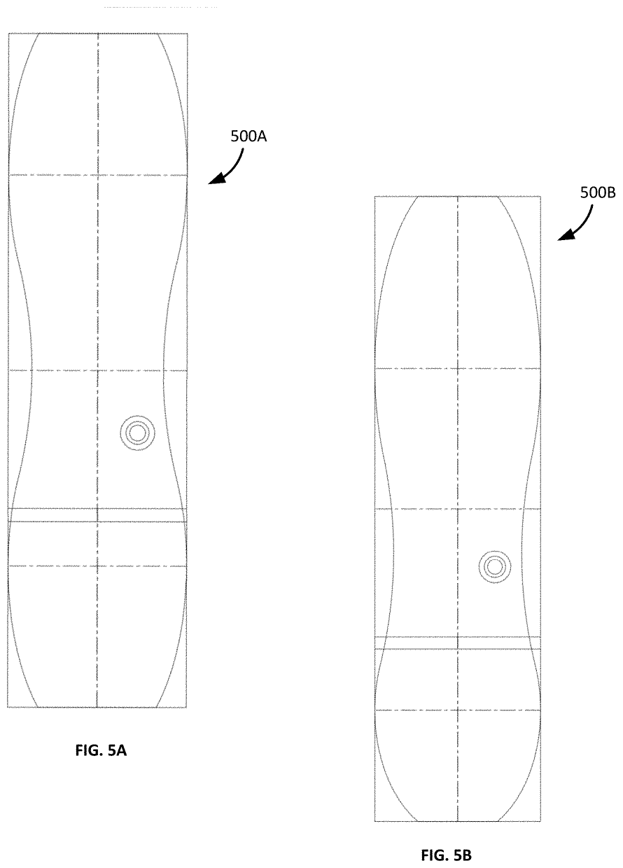

FIGS. 5A-B are schematic view of other profiles of the blood pressure cuff.

Various embodiments of the present invent on will now be described with reference to the appended drawings. It is to be appreciated that these drawings depict only some embodiments of the invention and are therefore not to be considered limiting of its scope.

### DETAILED DESCRIPTION

Despite the various improvements that have been made to blood pressure cuffs, conventional devices suffer from some shortcomings as described above.

Therefore, there is a need for further improvements to the devices, systems, and methods of manufacturing blood pressure cuffs. Among other advantages, the present disclosure may address one or more of these needs. To increase patient comfort, several non-rectangular blood pressure cuffs are disclosed, each having improvements on the prior art.

FIG. 1A-G are schematic bottom, top, perspective, front, back, and side views of a blood pressure cuff 100 according to a first embodiment of the present disclosure. Cuff 100 extends between proximal end 102 and distal end 104 and includes a body 105 having an expandable chamber for receiving a fluid (e.g., gas). Body 105 may include two layer of material that define the chamber 106 therein, the two layers defining an anterior surface 110 and a posterior surface 112 (FIGS. 1A-B). The chamber 106 is shown in FIG. 1B, and may generally be between 40% to 60% of the length of the cuff. In at least some examples, body 105 has a rounded piping 107 disposed around its perimeter as shown in the detailed view of FIG. 1A. This piping 107, though not shown, is a possible feature on all of the cuffs disclosed herein. In some examples, body 105 may include a fabric or cloth. In some examples, body 105 may be formed of an elastic material, such as spandex, or a non-woven synthetic material and may include a polyurethane material, or a latex-free material or combinations thereof. In at least some examples, X-STATIC Silver® brand of material may be woven into the fabric, the X-STATIC Silver® brand of material providing antimicrobial benefits to possibly prevent or decrease the risk of infection, or inhibit the growth of bacteria. X-STATIC Silver® brand of material may also be used to eliminate odor in the cuff. In at least some examples, the materials described above may be formed on, or form portions of the anterior surface, the posterior surface, or both surfaces.

Attachment means are used to couple one end of the cuff to another portion of the cuff to maintain it in a rolled condition. In one example, a VELCRO® brand hook-and-loop fastener system is used, where a hook region 116 is disposed on an anterior surface adjacent the proximal end 102 of the body, while an elongated fabric or cloth having a loop region 118 is disposed on a posterior surface adjacent the distal end 104. In at least some example, the loop region 118 is formed of a separate material attached to the body. Alternatively, the entire anterior surface of the body may include loops that are coupleable to the hook region 116. It will be understood that the hook and loop regions are interchangeable with one another. A hose connector 120 is disposed on the anterior surface 110 of the blood pressure cuff 100, and configured to mate with a hose to receive a gas into the chamber of the body.

As shown, the sides of the body are substantially non-linear. Specifically, sides of the body include a first set of concave curvatures C1,C2 adjacent the proximal end 102 approximately one ⅓ along the length of the body from the proximal end to the distal end, the curvatures C1,C2 being disposed opposite one another, a second set of concave curvatures C3,C4 adjacent the distal end 104, and enlarged convex curvatures C5,C6 disposed between the two sets of concave curvatures. Pairs of curvature may be symmetric. That is, C1 and C2 and may have a same first curvature, C3 and C4 may have a same second curvature, and C5 and C6 may have a same third curvature. As shown, the cuff may have a series of varying widths from the proximal end 102 to the distal end 104. In one example, cuff 100 may have a first width 1W1 of 3″, a second width 1W2 of 2 and 7/16″, a third width 1W3 of 3⅜″ and a fourth width 1W4 of 2 and 7/16″. It will be understood that the measurements provided for this embodiment, and in other examples, are presented by way of example to show one possible set of proportions of the widths of the various components of a cuff. Thus, the disclosed cuffs may have different measurements, and may be scaled up or down as desired based on the application. In some examples, cuff 100 may have a concave curvature at C1,C2 that decreases the width of the cuff by 5% to 20% when comparing 1W1 and 1W2, and the width may increase at 1W3 at C5,C6 by 5% to 10% as compared to 1W1. The width at 1W4 may be equal to the width at 1W2, or may be slightly smaller than 1W1 by decreasing by 5% to 20% when compared to 1W1.

Body may further include a brachial artery indicator 130 disposed on the anterior surface 110 slightly below the hooks region 116 (i.e., closer to the distal end 104 than the hooks region 116). Indicator 130 may help position the device on the patient's limb.

In use, the cuff 100 may be wrapped around the patient's limb (e.g., arm) as shown in FIG. 1H and regions 116 and 118 may be coupled together to maintain the cuff in the rolled condition around the patient's arm. A conduit or hose may be coupled to connector 120 to deliver a fluid or gas into the cuff. The curvatures of the cuff may sit adjacent key landmarks on the arm. For example, curvature C1 may sit adjacent the upper arm axilla, and curvature C2 may sit adjacent the antecubital. The curvatures may provide increased comfort to the patient and allow for greater flexion and/or degree of motion of the arm within the cuff.

Other configurations are also possible. For example, FIG. 2A-G are schematic bottom, top, perspective, front, back, and side views of a blood pressure cuff 200 according to a second embodiment of the present disclosure. Cuff 200 extends between proximal end 202 and distal end 204 and includes a body 205 having an expandable chamber 206 for receiving a fluid. Body 205 has an anterior surface 210 and a posterior surface 212 (FIGS. 2A-B), and may include any of the materials described above. Attachment means 216,218 may also be provided as described above with reference to hook and loop regions 116,118, and a hose connector 220 may be disposed on the anterior surface 210 of the blood pressure cuff 200.

As shown, the sides of the body are substantially non-linear. Specifically, sides of the body include a first set of concave curvatures D1,D2 adjacent the proximal end 202 disposed opposite one another, a second set of indentations or tapers D3,D4 adjacent the distal end 204, and a linear portion disposed therebetween. Pairs of curvature may be symmetric as previously described.

As shown, cuff 200 may have a series of varying widths from the proximal end 202 to the distal end 204. In one example, cuff 200 may have a first width 2W1 of 1 3/16″, a second width 2W2 of ¾″, a third width 2W3 of 1 3/16″ and a fourth width 2W4 of ¾″. In some examples, cuff 200 may have a concave curvature at D1,D2 that decreases the width of the cuff by 15% to 35% when comparing 2W1 and 2W2, and the width may increase at 2W3 to a width approximately equal to 2W1. The width at 2W4 at the end of the tapers D3,D4 may be approximately equal to the width at 2W2.

Body may further include a brachial artery indicator 230 disposed on the anterior surface 210 slightly below the hooks region 216 (i.e., closer to the distal end 204 than the hooks region 216). Indicator 230 may help position the device on the patient's limb. In use, the cuff 200 may be wrapped around the patient's limb and curvatures D1,D2 of the cuff 200 may sit adjacent key landmarks on the arm. For example, curvature D1 may sit adjacent the upper arm axilla, and curvature D2 may sit adjacent the antecubital. Additionally, tapers D3,D4 may allow for increased comfort when the cuff is wrapped over an arm.

A third embodiment is shown in FIGS. 3A-G. Cuff 300 extends between proximal end 302 and distal end 304 and includes a body having an expandable chamber 306 for receiving a fluid. The body has an anterior surface 310 and a posterior surface 312 (FIGS. 3A-B), and may include any of the materials described above. The main difference in this third embodiment is the curvatures E1-E6. Most notably, cuff 300 includes a pair of convex curvatures on each side (i.e. E1 and E5 on one side, and E2 and E6 on a second side), and a concave curvature (E3 on one side, and E4 on a second, opposing side) approximately halfway along the length of the cuff, the concave curvature being sandwiched between the convex curvatures.

As shown, the cuff may have a series of varying widths from the proximal end 302 to the distal end 304. In one example, cuff 300 may have a first width 3W1 of ⅜″, a second width 3W2 of 1⅜″, a third width 3W3 of 1¼″, a fourth width 3W4 of 1 7/16″, and a fourth width 3W5 of ¾″. In some examples, cuff 300 may have a convex curvature at E1,E2 that increases the width of the cuff by 50% to 400% when comparing 3W1 and 3W2, and the width may decrease at 3W3 at E3,E4 by 5% to 20% as compared to 3W2. The width at 3W4 may be equal to the width at 3W2, or may be slightly larger than 3W2 by increasing the width by 5% to 10% when compared to 3W2. The width at 3W5 may be equal to 3W1 or larger by 30% to 50%.

A fourth embodiment is shown in FIGS. 4A-G. Cuff 400 extends between proximal end 402 and distal end 404 and includes a body having an expandable chamber 406 for receiving a fluid. The body has an anterior surface 410 and a posterior surface 412 (FIGS. 4A-B), and may include any of the materials described above. The main difference in this fourth embodiment is the curvatures F1-F6. Most notably, cuff 400 includes a pair of convex curvatures on each side (i.e. F1 and F5 on one side, and F2 and F6 on a second side), and a concave curvature (F3 on one side, and F4 on a second, opposing side) approximately halfway along the length of the cuff, the concave curvature being sandwiched between the convex curvatures.

As shown, the cuff may have a series of varying widths from the proximal end 402 to the distal end 404. In one example, cuff 400 may have a first width 4W1 of 3″, a second width 4W2 of 5.5″, a third width 4W3 of 4.5″, a fourth width 4W4 of 5.5″, and a fifth width 4W5 of 3″. In at least some examples, cuff 400 is symmetric about both a vertical and a horizontal axis. Other profiles are also possible as shown in FIGS. 5A and 5B with cuffs 500A,500B.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A blood pressure cuff extending between a proximal end and a distal end comprising:
a body having a fillable chamber and a hose connector, the body having a pair of opposing non-linear sides having at least one first pair of concave curvatures formed approximately halfway along a length of the body from the proximal end to the distal end.

2. The blood pressure cuff of claim 1, wherein each of the first pair of concave curvatures has a same radius of curvature.

3. The blood pressure cuff of claim 1, further comprising a second pair of concave curvatures formed adjacent the distal end.

4. The blood pressure cuff of claim 3, further comprising a third pair of convex curvatures formed between the first pair of concave curvatures and the second pair of concave curvatures.

5. The blood pressure cuff of claim 1, further comprising a pair of tapered portions adjacent the distal end.

6. The blood pressure cuff of claim 1, further comprising linear portions disposed between the first pair of concave curvatures and the tapered portions.

7. The blood pressure cuff of claim 1, wherein the body includes X-STATIC Silver® brand of material.

8. The blood pressure cuff of claim 1, wherein the body includes a posterior surface and an anterior surface, and a pair of attachment means on each one of the posterior and anterior surfaces.

9. The blood pressure cuff of claim 8, further comprising an indicator disposed on the anterior surface of the body and disposed closer to the proximal end than the first pair of concave curvatures.

10. A blood pressure cuff extending between a proximal end and a distal end comprising:
a body having a fillable chamber and a hose connector, the body having a pair of opposing non-linear sides having at least one first pair of concave curvatures formed approximately halfway along a length of the body from the proximal end to the distal end, each of the first pair of concave curvatures has a same radius of curvature.

11. The blood pressure cuff of claim 10, wherein the body includes a posterior surface and an anterior surface, and pair of attachment means on each one of the posterior and anterior surfaces.

12. The blood pressure cuff of claim 11, further comprising an indicator disposed the anterior surface of the body and disposed closer to the proximal end than the first pair of concave curvatures.

13. The blood pressure cuff of claim 10, further comprising a second pair of concave curvatures formed adjacent the distal end.

14. The blood pressure cuff of claim 13, wherein each of the second pair of concave curvatures has a same radius of curvature.

15. The blood pressure cuff of claim 13, further comprising a third pair of convex curvatures formed between the first pair of concave curvatures and the second pair of concave curvatures.

16. The blood pressure cuff of claim 15, wherein each of the third pair of convex curvatures has a same radius of curvature.

17. The blood pressure cuff of claim 10, further comprising a pair of tapered portions adjacent the distal end.

18. The blood pressure cuff of claim 17, further comprising linear portions disposed between the first pair of concave curvatures and the tapered portions.

19. The blood pressure cuff of claim 10, further comprising a rounded piping disposed around the perimeter of the cuff.
